(19) United States
(12) Patent Application Publication
(10) Pub. No.: US 2005/0080062 A1
(43) Pub. Date: Apr. 14, 2005

(54) Breast cancer treatment regimen

(76) Inventor: Paul E. Goss, Toronto (CA)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/681,913

(22) Filed: Oct. 9, 2003

(51) Int. Cl. A61K 31/56; A61K 31/445; A61K 31/4178

(52) U.S. Cl. 514/171; 514/397; 514/408; 514/651; 514/319

(57) ABSTRACT

This invention relates to a method for preventing or delaying the progression of hormone receptor positive of hormone receptor unknown breast cancer in a patient by following estrogen antagonist therapy by subsequent therapy with an estrogen depleting agent prior to disease progression.

This invention relates to a treatment regimen for the treatment of breast cancer wherein estrogen antagonist (anti-estrogen) therapy is followed by therapy with an estrogen depleting agent prior to disease progression.

Breast cancer continues to recur indefinitely after diagnosis in loco-regional and distant sites despite the benefits of initial surgery, radiation and medical therapies. The pathogenesis of breast cancer is intimately related to estrogen and in patients, whose tumors are hormone receptor positive, substantial long term reductions in disease recurrence have been achieved by treatment with an estrogen antagonist, such as tamoxifen. However, about five years of postoperative therapy with an estrogen antagonist, such as tamoxifen, seems to be the optimal treatment period for reducing the odds of recurrence and death. It has been reported that no further benefit is achieved by continued treatment with an estrogen antagonist, but rather a paradoxical increase in breast cancer recurrences is associated with estrogen antagonist treatment for more than 5 years.

According to the Physician's Desk Reference, 57^{th} Edition (2003), the estrogen depleting agents anastrozole (ARIMIDEX®) marketed by ASTRAZENECA) and letrozole (FEMARA® marketed by NOVARTIS) have each been approved for the treatment of advanced breast cancer in postmenopausal women with disease progression following tamoxifen or anti-estrogen therapy.

The present invention is based on the theory that disease progression is delayed or prevented by treatment with an estrogen depleting agent if administered after estrogen antagonist therapy is withdrawn, but prior to disease progression. The hypothesis was confirmed by conducting a clinical study of the effects of treatment with the aromatase inhibitor letrozole in postmenopausal women who had completed five years of adjuvant tamoxifen treatment. Suprisingly, the results of the study demonstrated that following five years of treatment with tamoxifen by further treatment with the aromatase inhibitor markedly reduced breast cancer recurrance and new primary tumors. This finding marks a significant advance in the treatment options for the approximately one million women worldwide who are currently being treated with tamoxifen.

The present invention relates to a method for preventing or delaying the progression of hormone receptor positive or hormone receptor unknown breast cancer in a patient, which comprises following estrogen antagonist therapy by subsequent therapy with an estrogen depleting agent prior to disease progression.

Disease progression means recurrence of the primary disease (in the breast, chest wall, nodal or metastatic sites) or the development of contralateral breast cancer.

Hormone receptor positive and hormone receptor unknown means that the cancer cells test positive for the presence of estrogen or progestrone receptors or that the status of such receptors is unknown, respectively. Whether the cancer cells are hormone receptor positive is determined by methods known in the art.

Estrogen antagonists, also referred to as anti-estrogens, are competitive inhibitors of estradiol binding to the estrogen receptor. Estrogen antagonists and their administration for the treatment of breast cancer are known to those of skill in the art. Known estrogen antagonists include tamoxifen, fulvestrant, toremifene and raloxifene, and pharmaceutically effective salts thereof, especially tamoxifen and its pharmaceutically acceptable salts, particularly tamoxifen citrate.

Generally, the therapy with an estrogen antagonist is adjuvant therapy.

Estrogen depleting agents reduce serum estradiol levels in the patient. The aromatase (estrogen synthetase) inhibitors, which inhibit the enzyme that converts androgens to estrogens, are an especially important class of estrogen depleting agent. Aromatase inhibitors useful according the present invention include steroidal aromatase inhibitors, such as formestane and exemestane, and non-steroidal aromatase inhibitors, such as anastrozole, vorozole, letrozole and aminoglutethimide. Preferably a non-steroidal aromatase inhibitor, such as anastrozole or letrozole, is used. The use of such aromatase inhibitors for the treatment of hormone sensitive breast cancer is known to those of skill in the art, for example anastrozole is administered at a dose of one mg daily and letrozole is administered at a dose of 2.5 mg daily.

Generally, the therapy with the estrogen depleting agent is adjuvant therapy.

In accordance with the present invention, therapy with an estrogen antagonist refers to the standard treatment regimen with such agents for a period of time that such agents are expected to remain effective in preventing disease recurrence and/or death. Generally, therapy with an estrogen antagonist continues for up to about six years, for example from about six months to about six years, preferably about 4.5 years to about six years, optimally about 5 years. Accordingly, therapy with tamoxifen generally refers to administration of 20-40 mg of tamoxifen daily (30.4-60.8 mg of tamoxifen citrate daily) for a period of up to six years.

Therapy with an estrogen depleting agent refers to a treatment period during which the estrogen depleting agent has a positive effect, such as the period where there is no disease progression. Therapy with an estrogen depleting agent should continue for five years and could continue until disease progression or death.

In view of the discussion above, it is clear to one of skill that the present invention further relates to a method of improving the likelihood of disease-free survival or overall survival for a hormone receptor positive or hormone receptor unknown breast cancer patient who has been treated with tamoxifen, or a pharmaceutically acceptable salt thereof, which comprises subsequent therapy with an estrogen depleting agent prior to disease progression, especially wherein the estrogen depleting agent is an aromatase inhibitor, such as formestane, exemestane, anastrozole, vorozole, letrozole and aminoglutethimide, or a pharmaceutically acceptable salt thereof, or more particularly a non-steroidal aromatase inhibitor, especially anastrozole and letrozole, or a pharmaceutically acceptable salt thereof. The inventive method is particularly useful for treating patients who were treated with tamoxifen, or a pharmaceutically acceptable salt thereof, in an adjuvant setting for period of up to six years, particularly a period of from 4.5 to 6 years.

The present invention further relates to a packaged pharmaceutical composition of an aromatase inhibitor for the treatment of hormone receptor positive or hormone receptor unknown breast cancer in patients who have previously been treated with tamoxifen, or a pharmaceutically acceptable salt thereof, which includes advice that the likelihood of disease-free survival or overall survival could be improved by subsequent therapy with the aromatase inhibitor prior to disease progression, particularly wherein the aromatse inhibitor is anastrozole or letrozole.

### METHODS

Study Design

The study was a phase III randomized double-blind placebo-controlled study of letrozole in postmenopausal women with primary breast cancer completing five years of adjuvant tamoxifen. Patients were allocated to receive either letrozole 2.5 mg or placebo once daily by mouth for five years. Stratification factors at randomization were tumor receptor status (positive, unknown), lymph node status at diagnosis (negative, positive or unknown) and prior adjuvant chemotherapy (yes, no). The primary objective was disease-free survival (DFS). Secondary endpoints included overall survival (OS)(all cause mortality), contralateral breast cancer occurrence, quality of life and long term clinical and laboratory safety with special attention paid to cardiovascular morbidity and mortality and bone fractures. Adverse events were assessed by the NCI Common Toxicity Criteria version 2.0. Quality of life was assessed using the SF-36 and Menopause-Specific Quality of Life questionnaires. Companion studies assessed lipid profiles and changes in bone density.

Patient Population

All eligible patients had to be postmenopausal at randomization defined as: ≧50 years of age at the start of treatment with adjuvant tamoxifen; <50 years of age but considered postmenopausal at the time adjuvant tamoxifen was started; <50 years of age at tamoxifen initiation but having had a bilateral oophorectomy. Premenopausal women who were <50 years at the start of treatment with tamoxifen but who became amenorrheic during the course of adjuvant chemotherapy or during their treatment with tamoxifen and who remained so throughout the ensuing years of tamoxifen treatment and women with LH/FSH levels within post-menopausal limits were also eligible. Other eligibility included: histologically confirmed invasive breast cancer; tumor receptor positive or unknown (ER and/or PgR positive defined as a tumour receptor content of ≧10 fmol/mg protein, or receptor positive by ERICA or PgRICA); tamoxifen discontinued <3 months prior to enrolment; ECOG performance status 0,1 or 2 and a life expectancy of >5 years. Absence of metastatic disease prior to study enrolment had to be demonstrated if patients had abnormal blood work or symptoms of disease. Ineligibility included: concurrent use of investigational drugs; prior or concurrent malignancy other than skin cancer or carcinoma in situ of the cervix.

Concomitant medications prohibiting enrolment included: systemic hormone replacement therapy or SERMS (selective estrogen receptor modulators). Intermittent use of vaginal estrogens was permitted.

Study Procedures

All study participants were randomised. Study medication began within 5 working days of randomisation. Clinical evaluation, routine blood work, toxicity evaluation and mammography (annually only) occurred six monthly in year one and annually thereafter. Serious toxicities and deaths were reported within 24 hours. Treatment was discontinued for: serious intercurrent illness, unacceptable toxicity, disease recurrence and patient request. SF-36 and Menqol questionnaires were completed by a subset of patients at each visit. Fasting lipid samples and bone mineral density evaluations occurred in sub-sets of participants on the bone and lipid companion studies. Recurrence of disease was defined histologically, cytologically or by clinical and/or radiologic suspicion and dated by the first time of detection. Management of recurrent cancer was at the discretion of the treating physician. Patients developing any second malignancy other than non-melanoma skin cancer or carcinoma in-situ of the cervix had to discontinue study therapy.

Interim analyses and other decisions regarding early termination of the study were referred to an independent Data and Safety Monitoring Committee (DSMC) consisting of clinicians, patient representatives and statisticians who reviewed the study twice a year.

Statistical Analysis

Disease free survival, defined as the time from randomization to the time of recurrence of the primary disease (in the breast, chest wall, nodal or metastatic sites) or development of contralateral breast cancer, was the primary endpoint of the study. The sample size calculation assumed a 4 year disease free survival of 88% for patients on the placebo arm and a hazard ratio of 1.28, which represents 2.5% improvement in 4 year disease free survival from 88% to 90.5%. This required 4800 patients accrued over 4 years and followed for at least 2 years to observe 515 events before the final analysis. The study was closed in September 2002 with 5187 patients randomized, as additional patient entry was allowed to complete accrual to the bone substudy after the 4800 initial patients had been enrolled.

Two interim analyses were scheduled at 171 and 342 events respectively. Early termination of the study at the interim analyses was to be considered if the p-value of the stratified log-rank test was less than the nominal significance level calculated based on the number of events observed at the time of the interim analysis and from the Lan and DeMets alpha spending function with O'Brien-Fleming type boundaries to maintain the overall significance level of the study at a two-sided 0.05 level.

The required minimum number of events for the first interim analysis (171) was observed in March 2003. DFS and overall survival (OS), defined as the time from randomization to the time of death from any cause, were the two efficacy endpoints for the interim analysis. The stratified log-rank test was used to compare the DFS and OS between the treatment arms Chi-square test was used to compare toxicities between the two arms.

### RESULTS

Patient Population

A total of 5187 patients were randomized into the study from August 1998 until September 2002, 2593 on letrozole and 2594 on placebo. Thirty patients, (18 letrozole and 12 on placebo) whose baseline investigation forms were not received at the time of the database lock, were excluded from analyses. Thirty nine patients (19 letrozole; 20 placebo) were considered ineligible due to: improper time on, or off, prior tamoxifen, menopausal status, prior recurrence, prior or concurrent malignancy, inadequate primary surgery, receptor negative tumor, inadequate baseline investigations or simultaneous hormone therapy. Baseline patient characteristics are shown in Table 1. Standard baseline prognostic variables and pre-existing bone fractures, osteoporosis and cardiovascular disease were balanced between the arms. Median age of the patients was 62 years and 90% had a performance status ECOG 0. Within each arm there was a balance of patients who had prior mastectomy or lumpectomy, radiation and chemotherapy and who had node negative or positive disease. Ninety eight percent of the patients had known receptor positive tumors.

Patient Outcome

At the first analysis, 207 events (40% of the events required for final analysis) had occurred. Based on the Lan-DeMets alpha spending function with O'Brian-Fleming boundary, the trial would be stopped if the p-value of the stratified log-rank test for DFS were less than 0.00079. One thousand and forty eight patients were off protocol treatment at the time of the analysis. Reasons for coming off study included: treatment refusal (256 vs 254), toxicity (115 vs 93), progressive disease (44 vs 85) and other (98 vs 103) for letrozole and placebo respectively. Among the 207 events, 59 women on letrozole and 105 on placebo had metastatic disease recurrence only, 14 on letrozole and 26 on placebo developed contralateral breast cancer only, and 2 on letrozole and 1 on placebo developed both contralateral breast cancer and metastatic disease.

The 4 year DFS was respectively 93% (95% confidence interval from 90% to 95%) for patients on letrozole and 87% (95% confidence interval from 84% to 90%) for those on placebo. The hazard ratio of placebo to letrozole was 1.76 with a 95% confidence interval from 1.33 to 2.34. The p-value of the two sided log-rank test stratified by the stratification factors at randomization (receptor status, nodal status and prior adjuvant chemotherapy) was 0.000077.

A total of 72 patients had died at the time of data cut-off (30 on letrozole and 42 on placebo). The 4 year OS was 96% (95% confidence interval from 94% to 98%) for patients on letrozole and 94% (95% confidence interval from 91 to 96%) for those on placebo. The hazard ratio of patients on placebo versus those on letrozole was 1.36 with a 95% confidence interval from 0.85 to 2.17. The p-value of the two sided log-rank test stratified by the stratification factors (receptor status, nodal status and prior adjuvant treatment) at randomization was 0.20.

Based on the substantial reduction in DFS which exceeded the pre-specified stopping rule considerably, the observed reduction in mortality, and the very favorable toxicity profile of letrozole, the DSMC, the study chairman and the trial committee unanimously recommended unblinding all study participants and notifying them, their physicians and the public of these findings.

1. A method for preventing or delaying the progression of hormone receptor positive or hormone receptor unknown breast cancer in a patient, which comprises following estrogen antagonist therapy by subsequent therapy with an estrogen depleting agent prior to disease progression.

2. A method of claim 1 wherein the estrogen antagonist is selected from tamoxifen, fulvestrant, toremifene and raloxifene, or a pharmaceutically acceptable salt thereof.

3. A method of claim 2 wherein the estrogen antagonist is tamoxifen or pharmaceutically acceptable salt thereof.

4. A method of claim 2 wherein the estrogen depleting agent is an aromatase inhibitor.

5. A method of claim 3 wherein the estrogen depleting agent is an aromatase inhibitor.

6. A method of claim 4 wherein the estrogen depleting agent is an aromatase inhibitor selected from formestane, exemestane, anastrozole, vorozole, letrozole and aminoglutethimide, or a pharmaceutically acceptable salt thereof.

7. A method of claim 6 wherein the estrogen depleting agent is a non-steroidal aromatase inhibitor.

8. A method of claim 7 wherein the estrogen depleting agent is a non-steroidal aromatase inhibitor selected from anastrozole and letrozole, or a pharmaceutically acceptable salt thereof.

9. A method of claim 5 wherein the estrogen depleting agent is an aromatase inhibitor selected from formestane, exemestane, anastrozole, vorozole, letrozole and aminoglutethimide, or a pharmaceutically acceptable salt thereof.

10. A method of claim 9 wherein the estrogen depleting agent is a non-steroidal aromatase inhibitor.

11. A method of claim 10 wherein the estrogen depleting agent is a non-steroidal aromatase inhibitor selected from anastrozole and letrozole, or a pharmaceutically acceptable salt thereof.

12. A method of improving the likelihood of disease-free survival or overall survival for a hormone receptor positive or hormone receptor unknown breast cancer patient who has been treated with tamoxifen, or a pharmaceutically acceptable salt thereof, which comprises subsequent therapy with an estrogen depleting agent prior to disease progression.

13. A method of claim 12 wherein the estrogen depleting agent is an aromatase inhibitor.

14. A method of claim 13 wherein the estrogen depleting agent is an aromatase inhibitor selected from formestane, exemestane, anastrozole, vorozole, letrozole and aminoglutethimide, or a pharmaceutically acceptable salt thereof.

15. A method of claim 14 wherein the estrogen depleting agent is a non-steroidal aromatase inhibitor.

16. A method of claim 15 wherein the estrogen depleting agent is a non-steroidal aromatase inhibitor selected from anastrozole and letrozole, or a pharmaceutically acceptable salt thereof.

17. A method of claim 16 wherein the patient was treated with tamoxifen, or a pharmaceutically acceptable salt thereof, in an adjuvant setting for period of up to six years.

18. A method of claim 17 wherein the period of from 4.5 to 6 years.

19. A packaged pharmaceutical composition of an aromatase inhibitor for the treatment of hormone receptor positive or hormone receptor unknown breast cancer in patients who have previously been treated with tamoxifen, or a pharmaceutically acceptable salt thereof, which includes advice that the likelihood of disease-free survival or overall survival could be improved by subsequent therapy with the aromatase inhibitor prior to disease progression.

20. A method of claim 19 wherein the aromatase inhibitor is selected from anastrozole and letrozole, or a pharmaceutically acceptable salt thereof.
